(19) United States
(12) Patent Application Publication
(10) Pub. No.: US 2005/0095215 A1
(43) Pub. Date: May 5, 2005

(54) Antimicrobial shampoo compositions

(76) Inventor: Karl F. Popp, Schodack Landing, NY (US)

Correspondence Address: NATH & ASSOCIATES, 1030 15th STREET, NW, 6TH FLOOR, WASHINGTON, DC, 20005, US

(21) Appl. No.: 10/698,431

(22) Filed: Nov. 3, 2003

(51) Int. Cl. A61K 7/075; A61K 7/08

(52) U.S. Cl. 424/70.21; 424/70.22

(57) ABSTRACT

Topical pharmaceutical shampoos comprising an antimicrobial active ingredient, at least one surfactant, at least one hair conditioning agent, and at least one chelating agent. In a particular aspect, the antimicrobial active ingredient in the present inventive compositions has a concentration of degradation product(s) less than about 5% of the starting concentration of the active ingredient. These compositions are used for topical medical applications, particularly to treat various skin disorders.

### FIELD OF THE INVENTION

The present inventive subject matter relates to storage-stable topical pharmaceutical shampoos comprising an antimicrobial active ingredient. In a key aspect, the inventive shampoos are storage-stable in that the antimicrobial active ingredient maintains a concentration of degradation product(s) less than about 5% of the starting concentration of the active ingredient. These compositions are used for topical medical applications, particularly to treat various skin disorders.

### BACKGROUND OF THE INVENTION

Topical shampoo compositions incorporating antimicrobial active agents generally are known in the art. These antimicrobial shampoo compositions have been used for treating various microbial and fungal conditions of the head and scalp, such as seborrheic dermatitis and itchy, flaky scalp conditions, including stubborn dandruff. Current antimicrobial and antifungal shampoo products are mainly supplied in tubes or bottles and applied to the scalp by hand after wetting the hair.

However, these previously known shampoos containing an antimicrobial or antifungal agent have a low level of effectiveness. Accordingly, the results of using these shampoos are variable and unpredictable. In some cases, known antimicrobial shampoos seemed to improve and eradicate the scalp conditions, but in other cases the antimicrobials appeared to be ineffective on topical administration. They also commonly require an extended period of contact with the scalp before the shampoo is rinsed off. To remedy this low level of effectiveness, many prior shampoos have used an antimicrobial or antifungal agent in combination with another ingredient that serves as an additional active, as a penetration enhancer, or as an agent that stabilizes and enhances the activity of the antimicrobial or antifungal agent.

U.S. Pat. No. 5,665,776 describes a method for enhancing the therapeutic effect of a composition comprising an antifungal agent by combining an enhancing amount of a hydroxycarboxylic acid with the antifungal agent.

U.S. Pat. No. 5,919,438 describes methods for reducing or decelerating hair loss by applying to the scalp a shampoo composition containing at least one antifungal agent and at least one halogenated antibacterial agent. The halogenated antibacterial agent is taught as enhancing the effectiveness of the antifungal agent by inhibiting or preventing the growth of bacterial flora present at the surface of the epidermis rich in sebaceous glands.

U.S. Pat. No. 6,075,017 describes compositions for treating seborrheic dermatitis of the scalp comprising at least one cytotoxic agent and at least one antifungal agent. The cytotoxic agent is taught as providing a synergistic effect to the antifungal agent, allowing longer-term alleviation of the seborrheic dermatitis.

U.S. Pat. No. 6,284,234 describes a shampoo containing a micellar composition for enhancing the topical benefit of an antifungal agent through the use of 1-10% of a nonionic lipid.

U.S. Pat. No. 6,375,939 describes a shampoo composition containing at least one antifungal agent and at least one amphoteric polymer having at least one monomeric unit chosen from meth(acrylate) and meth(acrylamide) types. The amphoteric polymer increases the deposition of the antifungal agent, providing enhanced antifungal activity.

U.S. Pat. No. 6,383,523 describes a shampoo composition comprising greater than 4% of an acid component, hydrogen peroxide, and an antifungal agent. The acid component is included to exfoliate the skin while the hydrogen peroxide is included to cleanse the skin in order to facilitate the prevention, treatment, and management of skin conditions by the antifungal agent.

EP Patent No. 0,928,183 describes the use of 1-hydroxy-2-pyridone antifungal compounds for the production of a medicated shampoo for treating seborrheic dermatitis. However, this patent does not contemplate compositions maintaining a high purity level of the embodied antifungal agents in order to enhance their effectiveness and shelf life.

Accordingly, there remains a need in the art for antimicrobial shampoo products that do not rely on any additional ingredients to enhance the effect of the antimicrobial agent in treating various disorders of the scalp.

In this regard, the previously known antimicrobial shampoos did not recognize the benefits achieved by maintaining a high purity level of the antimicrobial agent with a low amount of degradates. This deficiency is overcome by the present formulations requiring high drug purity and low drug degradates, increasing the effectiveness and shelf life of the antimicrobial shampoo composition.

Accordingly, there remains a need in the art for antimicrobial shampoos useful in treating a variety of dermatological disorders that maintain a high purity level of the active antimicrobial agent(s) and a low level of degradates thereof. The present inventive subject matter addresses this need.

### SUMMARY OF THE INVENTION

The present inventive subject matter relates to a storage-stable topical pharmaceutical shampoo comprising:
about 0.5-8% by weight of an active ingredient comprising an antimicrobial agent or a pharmaceutically acceptable salt thereof;
about 0.5-30% by weight of at least one surfactant selected from the group consisting of an amphoteric surfactant, an anionic surfactant, and mixtures thereof;
about 0.01-1% by weight of at least one chelating agent;
about 40-90% by weight of purified water; and
sufficient amounts of at least one pH modifier selected from the group consisting of pharmaceutically acceptable acids, bases, and mixtures thereof to provide the pharmaceutical shampoo with an overall pH of about 3.0 to about 8.0;
wherein said active ingredient maintains a concentration of degradation product(s) less than about 5% of the starting concentration of said active ingredient.

In a preferred embodiment, the present inventive subject matter relates to a method of treating a skin or hair disorder in a mammal, comprising administering to skin or hair of a mammal in need thereof a therapeutically effective amount of a storage-stable topical pharmaceutical shampoo comprising:
about 0.5-8% by weight of an active ingredient comprising an antimicrobial agent or a pharmaceutically acceptable salt thereof;
about 0.5-30% by weight of at least one surfactant selected from the group consisting of an amphoteric surfactant, an anionic surfactant, and mixtures thereof;
about 0.01-1% by weight of at least one chelating agent;
about 40-90% by weight of purified water; and
sufficient amounts of at least one pH modifier selected from the group consisting of pharmaceutically acceptable acids, bases, and mixtures thereof to provide the pharmaceutical shampoo with an overall pH of about 3.0 to about 8.0;
wherein said active ingredient maintains a concentration of degradation product(s) less than about 5% of the starting concentration of said antimicrobial agent.

In another preferred embodiment, the present inventive subject matter relates to a storage-stable topical pharmaceutical shampoo comprising:
about 0.5-8% by weight of an active ingredient comprising ciclopirox or a pharmaceutically acceptable salt thereof;
about 0.5-30% by weight of at least one surfactant selected from the group consisting of an amphoteric surfactant, an anionic surfactant, and mixtures thereof;
about 0.01-1% by weight of at least one chelating agent;
about 40-90% by weight of purified water; and
sufficient amounts of at least one pH modifier selected from the group consisting of citric acid, sodium hydroxide, and mixtures thereof to provide the pharmaceutical shampoo with an overall pH of about 5.5 to about 7.0;
wherein said active ingredient maintains a concentration of degradation product(s) less than about 5% of the starting concentration of said active ingredient.

In yet another preferred embodiment, the present inventive subject matter relates to a method of treating a skin disorder in a mammal, comprising administering to a mammal in need thereof a therapeutically effective amount of a storage-stable topical pharmaceutical shampoo comprising:
about 0.5-8% by weight of an active ingredient comprising ciclopirox or a pharmaceutically acceptable salt thereof;
about 0.5-30% by weight of at least one surfactant selected from the group consisting of an amphoteric surfactant, an anionic surfactant, and mixtures thereof;
about 0.01-1% by weight of at least one chelating agent;
about 40-90% by weight of purified water; and
sufficient amounts of at least one pH modifier selected from the group consisting of citric acid, sodium hydroxide, and mixtures thereof to provide the pharmaceutical shampoo with an overall pH of about 5.5 to about 7.0;
wherein said active ingredient maintains a concentration of degradation product(s) less than about 5% of the starting concentration of said active ingredient.

### DETAILED DESCRIPTION OF THE INVENTION

### Definitions

As used herein, “antimicrobial” refers to any compound or composition which has activity against and/or reduces the number of microbes on a treated surface. Accordingly, and antimicrobial compound or composition has activity against various microbes such as bacteria, funguses, molds, and viruses. Antifungal compounds and compositions are considered a subset of antimicrobial compounds and compositions in this regard.

As used herein, “conditioner” or “conditioning agent” refers to a component which cleans, treats, softens, or otherwise affects the physical properties of a surface to which it is applied.

As used herein, “degradation products” refers to the product(s) produced by decomposition of one or more of the active ingredients of the present inventive compositions.

As used herein, an “extended period of time” refers to the shelf life of a composition of the present inventive subject matter, including time spent on the shelf at a pharmacy as well as the entire time period after sale of the composition during which the composition remains effective for the indicated use.

As used herein, the phrase “pharmaceutically acceptable salts” refers to salts of the active compound(s) which possess the same pharmacological activity as the active compound(s) and which are neither biologically nor otherwise undesirable. A salt can be formed with, for example, organic or inorganic acids. Non-limiting examples of suitable acids include acetic acid, acetylsalicylic acid, adipic acid, alginic acid, ascorbic acid, aspartic acid, benzoic acid, benzenesulfonic acid, bisulfic acid, boric acid, butyric acid, camphoric acid, camphorsulfonic acid, carbonic acid, citric acid, cyclopentanepropionic acid, digluconic acid, dodecylsulfic acid, ethanesulfonic acid, formic acid, fumaric acid, glyceric acid, glycerophosphoric acid, glycine, glucoheptanoic acid, gluconic acid, glutamic acid, glutaric acid, glycolic acid, hemisulfic acid, heptanoic acid, hexanoic acid, hippuric acid, hydrobromic acid, hydrochloric acid, hydroiodic acid, hydroxyethanesulfonic acid, lactic acid, maleic acid, malic acid, malonic acid, mandelic acid, methanesulfonic acid, mucic acid, naphthylanesulfonic acid, naphthylic acid, nicotinic acid, nitrous acid, oxalic acid, pelargonic, phosphoric acid, propionic acid, saccharin, salicylic acid, sorbic acid, succinic acid, sulfuric acid, tartaric acid, thiocyanic acid, thioglycolic acid, thiosulfuric acid, tosylic acid, undecylenic acid, and naturally and synthetically derived amino acids.

If organic bases are used, poorly volatile bases are preferably employed, for example low molecular weight alkanolamines such as ethanolamine, diethanolamine, N-ethylethanolamine, N-methyldiethanolamine, triethanolamine, diethylaminoethanol, 2-amino-2-methyl-n-propanol, dimethylaminopropanol, 2-amino-2-methylpropanediol, and triisopropanolamine. Ethanolamine is particularly preferred in this regard. Further poorly volatile bases which may be mentioned are, for example, ethylenediamine, hexamethylenediamine, morpholine, piperidine, piperazine, cyclohexylamine, tributylamine, dodecylamine, N,N-dimethyldodecylamine, stearylamine, oleylamine, benzylamine, dibenzylamine, N-ethylbenzylamine, dimethylstearylamine, N-methylmorpholine, N-methylpiperazine, 4-methylcyclohexylamine, and N-hydroxyethylmorpholine.

Salts of quaternary ammonium hydroxides such as trimethylbenzylammonium hydroxide, tetramethylammonium hydroxide, or tetraethylammonium hydroxide can also by used, as can guanidine and its derivatives, in particular its alkylation products. However, it is also possible to employ as salt-forming agents, for example, low molecular weight alkylamines such as methylamine, ethylamine, or triethylamine. Suitable salts for the compounds to be employed according to the present inventive subject matter are also those with inorganic cations, for example alkali metal salts, in particular sodium, potassium, or ammonium salts, alkaline earth metal salts such as, in particular, the magnesium or calcium salts, as well as salts with bi- or tetravalent cations, for example the zinc, aluminum, or zirconium salts. Also contemplated are salts with organic bases, such as dicyclohexylamine salts; methyl-D-glucamine; and salts with amino acids, such as arginine, lysine, and so forth. Also, the basic nitrogen-containing groups can be quaternized with such agents as lower alkyl halides, such as methyl, ethyl, propyl, and butyl chlorides, bromides, and iodides; dialkyl sulfates, such as dimethyl, diethyl, dibutyl, and diamyl sulfates; long chain halides, such as decyl, lauryl, myristyl, and stearyl chlorides, bromides, and iodides; asthma halides, such as benzyl and phenethyl bromides; and others. Water or oil-soluble or dispersible products are thereby obtained.

As used herein, “shampoo” refers to a cleanser composition capable of cleaning, conditioning, and/or treating living and non-living materials. Such non-living materials are meant to include wigs, hair toupees, and all the component parts thereof, such as rubber, plastic, adhesives, synthetic hair, and cloth.

As used herein, any “surface” to which the present antimicrobial compositions are applied encompasses physical areas related to the treatment of mammalian fungal diseases.

Other terms as used herein are meant to be defined by their well-known meanings in the art.

### Topical Antimicrobial Shampoos

The present inventive subject matter pertains to a storage-stable topical pharmaceutical shampoo comprising:
about 0.5-8% by weight of an active ingredient comprising an antimicrobial agent or a pharmaceutically acceptable salt thereof;
about 0.5-30% by weight of at least one surfactant selected from the group consisting of an amphoteric surfactant, an anionic surfactant, and mixtures thereof;
about 0.01-1% by weight of at least one chelating agent;
about 40-90% by weight of purified water; and
sufficient amounts of at least one pH modifier selected from the group consisting of pharmaceutically acceptable acids, bases, and mixtures thereof to provide the pharmaceutical shampoo with an overall pH of about 3.0 to about 8.0;
wherein said active ingredient maintains a concentration of degradation product(s) less than about 5% of the starting concentration of said active ingredient.

The present inventive antimicrobial shampoos are specifically formulated to possess the unique advantage of storage stability in that they maintain a high purity level and a low concentration of degradation products of the antimicrobial active ingredient. Accordingly, these antimicrobial shampoos do not require the essential presence of an additional ingredient to enhance the effectiveness of the antimicrobial agent on skin, hair, and scalp disorders.

Rather, the present inventive antimicrobial shampoos have an enhanced therapeutic effectiveness when compared with other antimicrobial shampoo products previously known in the art by virtue of a composition specifically tailored to maintain high drug purity and low levels of drug degradates. The selection of specific surfactants, conditioning agents, and chelating agents to form the compositions, as well as the preparation of an overall shampoo having a specific designated pH in the form of a designated emulsion, enables the present formulations to maintain a unique drug purity and the absence of inherent drug degradates.

Further, the high purity level and low concentration of degradation products permit the present inventive antimicrobial shampoos to have a longer shelf life when compared with other antimicrobial shampoo products previously known in the art.

In this regard, the present inventive compositions are able to maintain a low concentration of degradation product(s) of the active ingredients over an extended period of time. Accordingly, the present antimicrobial shampoos maintain a concentration of degradation product(s) less than about 5%, preferably less than about 2%, of the starting concentration of the active antimicrobial agent. This advantageous property was heretofore unknown in previous antimicrobial shampoo compositions.

Likewise, the present inventive antimicrobial shampoo compositions maintain a purity level of at least 95%, preferably at least 98%, of the antimicrobial active ingredient over an extended period of time. This advantageous property was similarly unknown in the field of antimicrobial shampoo technology.

The present inventive compositions containing the low level of degradative products and high purity level of active antimicrobial agent produce a greater medical effect than would be expected based on results obtained from antimicrobial shampoo products previously known in the art.

Further, the ability of the present compositions to exhibit a greater antimicrobial effect without resorting to an additional, enhancing agent represents a significant improvement over the compositions previously known in the art. This represents a significant advantage over prior antimicrobial shampoos. As fewer active ingredients are present in a composition, the chances of a patient having an adverse reaction to the composition will decrease. For example, the present inventive antimicrobial shampoos are expected to irritate the skin of a lower percentage of patients than do the previous antimicrobial shampoo compositions containing an effect-enhancing agent in addition to the antimicrobial agent.

The present inventive compositions are preferably formed as a clear solution. Accordingly, the pH of the aqueous phase, and of the final composition, is adjusted to range from about 3.0 to about 8.0. In a preferred embodiment, the pH of the final composition is adjusted to range from about 5.5 to about 7.0. In a particularly preferred embodiment, the pH of the final composition is adjusted to about 6.5.

Antimicrobial Agents

The present inventive storage-stable topical antimicrobial shampoo compositions comprise about 0.5 to about 8% by weight of an antimicrobial agent or a pharmaceutically acceptable salt thereof. In a particularly preferred embodiment, the present inventive compositions comprise about 1 to about 5% by weight of the antimicrobial agent or a pharmaceutically acceptable salt thereof. In a most preferred embodiment, the present inventive compositions comprise about 1.5 to about 3% by weight of the antimicrobial agent or a pharmaceutically acceptable salt thereof.

It is an essential aspect of the present inventive compositions that they maintain a purity level of at least 95%, preferably at least 98%, of the antimicrobial agent over an extended period of time. Likewise, it is critical that the present inventive compositions maintain a low concentration of degradation product(s) of the antimicrobial agent, namely less than about 5%, preferably less than about 2%, of the starting concentration of the antimicrobial agent over an extended period of time.

In a preferred embodiment, the antimicrobial agents used in the present compositions possess anti-inflammatory properties. Accordingly, the present shampoo compositions possess anti-inflammatory properties as well. In particular, the antimicrobial agents, and thus the antimicrobial compositions, possess activity against microbes selected from the group consisting of gram positive bacteria, gram negative bacteria, funguses, molds, viruses, and combinations thereof.

Particularly preferred antimicrobial agents useful in the present inventive compositions are those having the formula I: 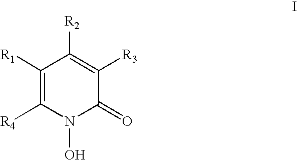
or a pharmaceutically acceptable salt thereof, wherein:
R₁, R₂, and R₃, which are identical or different, are H or alkyl having 1 to 4 carbon atoms, and R₄ is a saturated hydrocarbon radical having 6 to 9 carbon atoms or a radical of formula II: 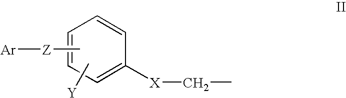
   where:
X is S or O;
Y is selected from the group consisting of H, 1 or 2 identical halogen atoms, and a mixture of 2 different halogen atoms;
Z is selected from the group consisting of a single bond and a bivalent radical comprising O, S, CR₂ where R₂ is H or (C₁-C₄)-alkyl, or from 2 to 10 carbon atoms linked in the form of a chain, which optionally further comprises one or more of the following:
   (i) a carbon-carbon double bond, or
   (ii) O, S, or a mixture thereof, wherein if 2 or more O or S atoms or a mixture thereof are present, each O or S atom is separated by at least 2 carbon atoms; and,
in any of the foregoing bivalent radicals, free valences of the carbon atoms of said bivalent radical are saturated by H, (C₁-C₄)-alkyl, or a mixture thereof; and
Ar is an aromatic ring system having one or two rings that can be substituted by one, two, or three radicals, which may be identical or different, which are selected from the group consisting of halogen, methoxy, (C₁-C₄)-alkyl, trifluoromethyl, or trifluoromethoxy. These compounds are preferably present in the free or in the salt form.

In the radical “Z”, the carbon chain members are preferably CH₂ groups. If the CH₂ groups are substituted by C₁-C₄ alkyl groups, CH₃ and C₂H₅ are preferred substituents. Exemplary radicals “Z” are:
—O—, —S—, —CH₂—, —(CH₂)ₘ— (m=2-10), —C(CH₃)₂—, —CH₂O—, —OCH₂—, —CH₂S—, —SCH₂—, —SCH(C₂H₅)—, —CH═CH—CH₂O—, —OCH₂CH═CHCH₂O—, —OCH₂CH₂O—, —OCH₂CH₂CH₂O—, —SCH₂CH₂CH₂S—, —SCH₂CH₂CH₂CH₂O—, —SCH₂CH₂OCH₂CH₂O—, —SCH₂CH₂OCH₂CH₂OCH₂CH₂S—, and —SCH₂C(CH₃)₂CH₂S—.

In the formula I, the hydrocarbon radical R₄ is preferably an alkyl or cyclohexyl radical which can also be bonded to the pyridone ring via a methylene or ethylene group or can contain an endomethyl group. R₄ can also be an aromatic radical which, however, is preferably bonded to the pyridone radical via at least one aliphatic carbon atom.

Preferred, non-limiting examples of the antimicrobial agent of formula I useful in the present inventive shampoo compositions are those selected from the group consisting of:
6-[4-(4-chlorophenoxy)-phenoxymethyl]-1-hydroxy-4-methyl-2-pyridone, 6-[4-(2,4-dichlorophenoxy)phenoxymethyl]-1-hydroxy-4-methyl-2-pyridone, 6-(biphenyl-4-oxymethyl)-1-hydroxy-4-methyl-2-pyridone, 6-(4-benzyl-phenoxymethyl)-1-hydroxy-4-methyl-2-pyridone, 6-[4-(4-chlorophenoxy)phenoxymethyl]-1-hydroxy-3,4-dimethyl-2-pyridone, 6-[4-(2,4-dichlorobenzyl)phenoxymethyl]-1-hydroxy-4-3,4-dimethyl-2-pyridone, 6-[4-cinnamyloxyphenoxymethyl]-1-hydroxy-4-methyl-2-pyridone, 1-hydroxy-4-methyl-6-[4-(4-trifluoromethylphenoxy)phenoxymethyl]-2-pyridone, 1-hydroxy-4-methyl-6-cyclohexyl-2-pyridone, 1-hydroxy-4-methyl-6-(2,4,4-trimethylpentyl)-2-pyridone, 1-hydroxy-4-methyl-6-n-hexyl-, -6-iso-hexyl-, -6-n-heptyl-, or -6-isoheptyl-2-pyridone, 1-hydroxy-4-methyl-6-octyl- or -6-isooctyl-2-pyridone, 1-hydroxy-4-methyl-6-cyclohexylmethyl- or -6-cyclohexylethyl-2-pyridone, where the cyclohexyl radical can in each case also carry a methyl radical, 1-hydroxy-4-methyl-6-(2-bicyclo-[2,2,1]heptyl)-2-pyridone, 1-hydroxy-3,4-dimethyl-6-benzyl- or -6-dimethylbenzyl-2-pyridone, 1-hydroxy-4-methyl-6-(β-phenylethyl)-2-pyridone, a pharmaceutically acceptable salt thereof, and a mixture thereof.

In a particularly preferred embodiment, the antimicrobial agent of formula I useful in the present inventive shampoo compositions is selected from the group consisting of:
6-[4-(4-chlorophenoxy)-phenoxymethyl]-1-hydroxy-4-methyl-2-pyridone, 1-hydroxy-4-methyl-6-cyclohexyl-2-pyridone, 1-hydroxy-4-methyl-6-(2,4,4-trimethylpentyl)-2-pyridone, a pharmaceutically acceptable salt thereof, and a mixture thereof.

In a most preferred embodiment, the antimicrobial agent of formula I used in the present inventive shampoo compositions is 1-hydroxy-4-methyl-6-cyclohexyl-2-pyridone (Ciclopirox) or a pharmaceutically acceptable salt thereof. The ciclopiroxolamine salt is particularly preferred in this regard.

Antimicrobial agents other than those falling within formula I above are additionally contemplated as useful in the present inventive antimicrobial shampoo compositions. Included among these other antimicrobial agents are those selected from the group consisting of imidazoles, allylamines, triazoles, glucan synthase inhibitors, chitin synthase inhibitors, polyenes, griseofulvin, morpholine derivatives, triazines, pyrimidines, any other antimicrobial azole, pharmaceutically acceptable salts thereof, and mixtures thereof. Other antimicrobial agents known in the art as effective upon topical administration to a patient are further contemplated as effective within the present inventive shampoo compositions.

In a preferred embodiment, these other antimicrobial agents are those selected from the group consisting of amorolfine, amphotericin B, butoconazole, chloroxine, cilofungin, chlordantoin, clotrimazole, econazole, faeriefungin, fezatione, fluconazole, flucytosine, fungimycin, haloprogin, itraconazole, ketoconazole, miconazole, naftifine, nikkomycin Z, nystatin, oxiconazole, pyrido[3,4-e]-1,2,4-triazine, pyrrolnitrin, salicylic acid, sulconazole, terbinafine, terconazole, thiabendazole, ticlatone, tolnaftate, triacetin, zinc and sodium pyrithione, a pharmaceutically acceptable salt thereof, and a mixture thereof.

In a particularly preferred embodiment, the other antimicrobial agent is selected from the group consisting of clotrimazole, econazole, fluconazole, ketoconazole, lamisol, miconazole, naftifine, oxiconazole, sulconazole, terbinafine, a pharmaceutically acceptable salt thereof, and a mixture thereof. Terbinafine or a pharmaceutically acceptable salt thereof is especially preferred in this regard.

Surfactants

The present inventive storage-stable topical antimicrobial shampoo compositions further comprise about 0.5 to about 30% by weight of at least one surfactant. In a particularly preferred embodiment, the present inventive compositions comprise about 12 to about 22% by weight of the at least one surfactant.

The selection of specific surfactant(s) in the specifically designated weight amounts helps provide the enhanced therapeutic effectiveness of the present inventive antimicrobial shampoos and maintenance of reduced amounts of active ingredient degradates when compared with other antimicrobial shampoo products previously known in the art.

The at least one surfactant useful in the present inventive antimicrobial shampoos is preferably selected from the group consisting of an amphoteric surfactant, an anionic surfactant, and mixtures thereof. In a particularly preferred embodiment, the present inventive compositions comprise at least one amphoteric and at least one anionic surfactant. When used in combination with an anionic surfactant, amphoteric surfactants have a synergistically enhanced foaming behavior, thickenability, and skin and eye mucous membrane tolerability.

Preferred, non-limiting examples of amphoteric surfactants useful in the present inventive antimicrobial shampoo compositions are those selected from the group consisting of alkyl betaines, alkylamidobetaines, aminopropionates, iminodipropionates, aminoglycinates, imidazolinium betaines, sulfobetaines, and mixtures thereof.

Specific, non-limiting examples of preferred amphoteric surfactants useful in the present inventive antimicrobial shampoos are those selected from the group consisting of sodium 3-dodecyl-aminopropionate, sodium 3-dodecylaminopropane sulfonate, sodium lauroamphoacetate, coco dimethyl carboxymethyl betaine, cocoamidopropyl betaine, cocobetaine, lauryl amidopropyl betaine, oleyl betaine, lauryl dimethyl carboxymethyl betaine, lauryl dimethyl alphacarboxyethyl betaine, cetyl dimethyl carboxymethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, lauryl bis-(2-hydroxypropyl)alpha-carboxyethyl betaine, oleamidopropyl betaine, coco dimethyl sulfopropyl betaine, stearyl dimethyl sulfopropyl betaine, lauryl dimethyl sulfoethyl betaine, lauryl bis-(2-hydroxyethyl)sulfopropyl betaine, and mixtures thereof.

In a most preferred embodiment, the present inventive antimicrobial shampoo compositions contain the amphoteric surfactant cocoamidopropyl betaine.

Similarly, preferred, non-limiting examples of anionic surfactants useful in the present inventive antimicrobial shampoo compositions are those selected from the group consisting of alkyl sulfates, alkyl ethoxylated sulfates, beta-alkyloxy alkane sulfonates, alkyl ether sulfates, alkyl glyceryl ether sulfonates, alkyl ether carboxylates, acyl isethionates, acyl sarcosinates, acyl taurines, succinates, alkali metal, ammonium, or alkanolammonium salts thereof, and mixtures thereof.

Specific, non-limiting examples of preferred anionic surfactants useful in the present inventive antimicrobial shampoos are those selected from the group consisting of ammonium lauryl sulfate, sodium lauryl sulfate, ammonium laureth sulfate, sodium laureth sulfate, alkyl glyceryl ether sulfonate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, sodium and ammonium salts of coconut alkyl triethylene glycol ether sulfate; tallow alkyl triethylene glycol ether sulfate, tallow alkyl hexaoxyethylene sulfate, disodium N-octadecylsulfosuccinnate, disodium lauryl sulfosuccinate, diammonium lauryl sulfosuccinate, tetrasodium N-(1,2-dicarboxyethyl)-N-octadecylsulfosuccinnate, diamyl ester of sodium sulfosuccinic acid, dihexyl ester of sodium sulfosuccinic acid, dioctyl esters of sodium sulfosuccinic acid, docusate sodium, and mixtures thereof.

In a most preferred embodiment, the present inventive antimicrobial shampoo compositions contain the anionic surfactant triethylamine lauryl sulfate.

It is further contemplated as within the scope of the presently claimed invention that additional surfactant(s) may be present with the anionic and/or amphoteric surfactants in the present inventive antimicrobial shampoos so long as the other surfactant(s) help maintain a purity level of at least 95% and a concentration of degradation product(s) less than about 5% of the antimicrobial agent over an extended period of time. These additional surfactant(s) can include nonionic and cationic surfactants.

Non-limiting examples of preferred cationic surfactants include those selected from the group consisting of behenyl trimethyl ammonium chloride, bis(acyloxyethyl)hydroxyethyl methyl ammonium methosulfate, cetrimonium bromide, cetyl trimethyl ammonium chloride, cocamido propylamine oxide, distearyl dimethyl ammonium chloride, ditallowdimonium chloride, guar hydroxypropyltrimonium chloride, lauralkonium chloride, lauryl dimethylamine oxide, lauryl dimethylbenzyl ammonium chloride, lauryl polyoxyethylene dimethylamine oxide, lauryl trimethyl ammonium chloride, lautrimonium chloride, methyl-1-oleyl amide ethyl-2-oleyl imidazolinium methyl sulfate, picolin benzyl ammonium chloride, polyquaternium, stearalkonium chloride, sterayl dimethylbenzyl ammonium chloride, stearyl trimethyl ammonium chloride, trimethylglycine, and mixtures thereof.

Non-limiting examples of preferred nonionic surfactants include those selected from the group consisting of polyoxyethylene fatty acid esters, sorbitan esters, cetyl octanoate, cocamide DEA, cocamide MEA, cocamido propyl dimethyl amine oxide, coconut fatty acid diethanol amide, coconut fatty acid monoethanol amide, diglyceryl diisostearate, diglyceryl monoisostearate, diglyceryl monolaurate, diglyceryl monooleate, ethylene glycol distearate, ethylene glycol monostearate, ethoxylated castor oil, glyceryl monoisostearate, glyceryl monolaurate, glyceryl monomyristate, glyceryl monooleate, glyceryl monostearate, glyceryl tricaprylate/caprate, glyceryl triisostearate, glyceryl trioleate, glycol distearate, glycol monostearate, isooctyl stearate, lauramide DEA, lauric acid diethanol amide, lauric acid monoethanol amide, lauric/myristic acid diethanol amide, lauryl dimethyl amine oxide, lauryl/myristyl amide DEA, lauryl/myristyl dimethyl amine oxide, methyl gluceth, methyl glucose sesquistearate, oleamide DEA, PEG-distearate, polyoxyethylene butyl ether, polyoxyethylene cetyl ether, polyoxyethylene lauryl amine, polyoxyethylene lauryl ester, polyoxyethylene lauryl ether, polyoxyethylene nonylphenyl ether, polyoxyethylene octyl ether, polyoxyethylene octylphenyl ether, polyoxyethylene oleyl amine, polyoxyethyelen oleyl cetyl ether, polyoxyethylene oleyl ester, polyoxyethylene oleyl ether, polyoxyethylene stearyl amine, polyoxyethylene stearyl ester, polyoxyethylene stearyl ether, polyoxyethylene tallow amine, polyoxyethylene tridecyl ether, propylene glycol monostearate, sorbitan monolaurate, sorbitan monooleate, sorbitan monopalmitate, sorbitan monostearate, sorbitan sesquioleate, sorbitan trioleate, stearamide DEA, stearic acid diethanol amide, stearic acid monoethanol amide, laureth-4, and mixtures thereof.

In a particularly preferred embodiment, the present inventive antimicrobial shampoos contain at least one surfactant selected from the group consisting of cocamidopropyl betaine, triethylamine lauryl sulfate, and mixtures thereof.

Chelating Agents

The present inventive storage-stable topical antimicrobial shampoo compositions further comprise about 0.01 to about 1% by weight of at least one chelating agent. The selection of specific chelating agent(s) in the specifically designated weight amounts helps provide the enhanced therapeutic effectiveness of the present inventive antimicrobial shampoos and maintenance of reduced amounts of active ingredient degradates when compared with other antimicrobial shampoo products previously known in the art.

Preferred non-limiting examples of chelating agents useful in the present inventive compositions are those selected from the group consisting of EDTA, disodium edetate, trans-1,2-diaminocyclohexane-N,N,N′,N′-tetraaceticacid monohydrate, N,N-bis(2-hydroxyethyl)glycine, 1,3-diamino-2-hydroxypropane-N,N,N′,N′-tetraacetic acid, 1,3-diaminopropane-N,N,N′,N′-tetraacetic acid, ethylenediamine-N,N′-diacetic acid, ethylenediamine-N,N′-dipropionic acid, ethylenediamine-N,N′-bis(methylenephosphonic acid), N-(2-hydroxyethyl)ethylenediamine-N,N′,N′-triacetic acid, ethylenediamine-N,N,N′,N′-tetrakis(methylenephosponic acid), O,O′-bis(2-aminoethyl)ethyleneglycol-N,N,N′,N′-tetraacetic acid, N,N-bis(2-hydroxybenzyl)ethylenediamine-N,N-diacetic acid, 1,6-hexamethylenediamine-N,N,N′,N′-tetraacetic acid, N-(2-hydroxyethyl)iminodiacetic acid, iminodiacetic acid, 1,2-diaminopropane-N,N,N′,N′-tetraacetic acid, nitrilotriacetic acid, nitrilotripropionic acid, nitrilotris(methylenephosphoric acid), 7,19,30-trioxa-1,4,10,13,16,22,27,33-octaazabicyclo[11,11,11]pentatriacontane hexahydrobromide, triethylenetetramine-N,N,N′,N″,N′″,N′″-hexaacetic acid, pharmaceutically acceptable salts thereof, and mixtures thereof.

In a most preferred embodiment, the present inventive antimicrobial shampoo compositions contain the chelating agent disodium edetate.

pH Modifiers

The present inventive storage-stable topical antimicrobial shampoo compositions further contain sufficient amounts of at least one pH modifier to ensure that the shampoo composition has a final pH of about 3.0 to about 8.0. The preparation of an overall shampoo having this specific pH in the form of a designated emulsion conveys the unique drug purity and drug degradate characteristics to the present inventive antimicrobial shampoos.

The pH modifiers useful in the present inventive compositions include salts, organic acids, inorganic bases and organic bases and the like. Preferred non-limiting examples of pH modifiers useful to impart the desired pH to the present inventive compositions are those selected from the group consisting of sodium hydroxide, citric acid, hydrochloric acid, acetic acid, phosphoric acid, succinic acid, sodium hydroxide, potassium hydroxide, ammonium hydroxide, magnesium oxide, calcium carbonate, magnesium carbonate, magnesium aluminum silicates, malic acid, potassium citrate, sodium citrate, sodium phosphate, lactic acid, gluconic acid, tartaric acid, 1,2,3,4-butane tetracarboxylic acid, fumaric acid, diethanolamine, monoethanolamine, sodium carbonate, sodium bicarbonate, triethanolamine, and a mixture thereof. The pH modifiers sodium hydroxide and citric acid are most preferred in this regard.

Conditioning Agents

The present inventive storage-stable topical antimicrobial shampoo compositions may further comprise about 0.1 to about 5% by weight of at least one conditioning agent. In a preferred embodiment, the conditioning agent is a hair conditioning agent. In a particularly preferred embodiment, the present inventive compositions comprise about 0.5 to about 2.5% by weight of the at least one hair conditioning agent.

The selection of specific conditioning agent(s) in the specifically designated weight amounts helps provide the enhanced therapeutic effectiveness of the present inventive antimicrobial shampoos when compared with other antimicrobial shampoo products previously known in the art. This enhanced effectiveness is in part achieved since the type and amount of conditioning agent(s) present help permit the maintenance of high drug purity and low levels of drug degradates.

Preferred conditioning agents useful herein affect the physical properties of a surface to which the present antimicrobial shampoo compositions are applied. These surfaces include those selected from the group consisting of populated hair, hair follicles, a surface contiguous to or in close proximity to hair, sweat glands, sebaceous glands, and combinations thereof. Preferred non-limiting examples of hair conditioning agents in this regard are those selected from the group consisting of a silicone compound, a quaternary ammonium compound, a fatty compound, a lanolin or a derivative thereof, and mixtures thereof.

Specific, non-limiting examples of preferred hair conditioning agents useful in the present inventive antimicrobial shampoos are those selected from the group consisting of cetrimonium chloride, ethoxylated polyethylene glycol lanolin, SILQUAT Q-100, SILQUAT Q-200, SILQUAT Q-300. SILQUAT-400, dimethyldiallylammonium chloride homopolymer, copolymers of acrylamide and dimethyldiallylammonium chloride, lauryl dimethyl ammonium-substituted epoxide, guar hydroxypropyltrimonium chloride, PEG Olealmonium Chloride, PEG Cocomonium Chloride, PEG Cocomonium Chloride, PEG Stearmonium Chloride, PEG Tallowmonium Chloride, stearamidopropyldimethylamine, stearamidopropyldiethylamine, stearamidoethyldiethylamine, stearamidoethyldimethylamine, paimitamidopropyldimethylamine, paimitamidopropyldiethylamine, palmitamidoethyldiethylamine, palmitamidoethyldimethylamine, behenamidopropyldimethylamine, behenamidopropyldiethylamine, behenamidoethyldiethylamine, behenamidoethyldimethylamine, arachidamidopropyldimethylamine, arachidamidopropyldiethylamine, arachidamidoethyldiethylamine, arachidamidoethyldimethylamine, diethylaminoethylstearamide, dimethylstearamine, dimethylsoyamine, soyamine, myristylamine, tridecylamine, ethylstearylamine, N-tallowpropane diamine, ethoxylated stearylamine, dihydroxyethylstearylamine, arachidylbehenylamine, laurtrimonium chloride, lauralkonium chloride, steartrimonium chloride, tallowtrimonium chloride, cetylpyridinium chloride, 2-ethylhexylamine, dodecylamine, dodecyl dimethylamine, hexadecyl dimethylamine, oleyl dimethylamine, cetyl dimethylamine, myristyl dimethylamine, oleyl amine, cocamine, and mixtures thereof.

In a most preferred embodiment, the present inventive antimicrobial shampoo compositions contain the hair conditioning agents cetrimonium chloride and ethoxylated polyethylene glycol lanolin.

Additional Ingredients

The present inventive antimicrobial shampoo compositions may further comprise one or more of several additional excipients commonly known to those of ordinary skill in the art as useful in topical compositions. Several non-limiting examples of such additional excipients include humectants, inorganic salts, fragrances, dyes, hair colorants, foam stabilizers, preservatives, water softening agents, thickeners, and mixtures thereof.

Non-limiting examples of specific humectants useful in the present inventive compositions include glycerin, butylene glycol, propylene glycol, sorbitol, and triacetin.

Non-limiting examples of specific dyes useful in the present inventive compositions include any of the FD&C or D&C dyes.

Non-limiting examples of specific hair colorants useful in the present inventive compositions include hydrogen peroxide, perborate salts, and persulfate salts.

Non-limiting examples of specific preservatives useful in the present inventive compositions include methylparaben, benzalkonium chloride, propylparaben, benzoic acid, EDTA, phenolic acid, sorbic acid, benzyl alcohol, isopropyl alcohol, benzethonium chloride, bronopol, butylparaben, cetrimide, chlorhexidine, chlorobutanol, chlorocresol, cresol, ethylparaben, glycerol, imidurea, phenol, phenoxyethanol, phenylethyl alcohol, potassium sorbate, propylene glycol, sodium benzoate, sodium propionate, sorbic acid, thimerosol, and mixtures thereof. A particularly preferred preservative in this regard is methylparaben.

A preferred, non-limiting example of a water softening agent useful in the present inventive compositions is editic acid.

Non-limiting examples of specific thickeners useful in the present inventive compositions include methylcellulose, hydroxybutyl methylcellulose, hydroxypropylcellulose, hydroxypropyl methylcellulose, hydroxyethyl ethylcellulose, hydroxyethylcellulose, di(hydrogenated tallow)phthalic acid amide, crosslinked maleic anhydride-methyl vinyl ether copolymer, guar gum, xanthan gum, gum arabic, lauramide MEA, and mixtures thereof.

The present antimicrobial shampoos can preferably be in the form of a shampoo physically similar to, or in the form of, a lotion, cream, solution, suspension, or dispersion.

### Methods of Treatment

The present inventive subject matter additionally pertains to a method of treating a skin or hair disorder in a mammal, comprising administering to skin or hair of a mammal in need thereof a therapeutically effective amount of a storage-stable topical pharmaceutical shampoo comprising:
about 0.5-8% by weight of an active ingredient comprising an antimicrobial agent or a pharmaceutically acceptable salt thereof;
about 0.5-30% by weight of at least one surfactant selected from the group consisting of an amphoteric surfactant, an anionic surfactant, and mixtures thereof;
about 0.01-1% by weight of at least one chelating agent;
about 40-90% by weight of purified water; and
sufficient amounts of at least one pH modifier selected from the group consisting of pharmaceutically acceptable acids, bases, and mixtures thereof to provide the pharmaceutical shampoo with an overall pH of about 3.0 to about 8.0;
   wherein said active ingredient maintains a concentration of degradation product(s) less than about 5% of the starting concentration of said antimicrobial agent.

Preferred examples of skin or hair that can be treated according to these methods are selected from the group consisting of populated hair, hair follicles, a surface contiguous to or in close proximity to hair, sweat glands, sebaceous glands, and combinations thereof. The treated skin or hair is natural or synthetic skin or hair.

In preferred embodiments, the administration of the antimicrobial shampoo cleans hair or scalp of the mammal to which it is applied. Accordingly, the administration of the shampoo reduces the number of microbes, preferably pathogenic microbes, on the hair or scalp of the mammal to which it is applied. The microbes that can be acted on by the present antimicrobial shampoos are selected from the group consisting of bacteria, funguses, molds, viruses, and combinations thereof.

Preferred examples of bacteria treatable with the present antimicrobial shampoos are gram positive bacteria, gram negative bacteria, and combinations thereof. Specific, non-limiting examples of such gram positive bacteria are those selected from the group consisting of Streptococcus sp., Micrococcus sp., Staphylococcus sp., Bacillus sp., and combinations thereof.

Preferred, non-limiting examples of such Streptococcus sp. are those selected from the group consisting of S. viridans, S. agalactiae, S. pyogenes, S. faecalis, S. durans, S. faecium, S. mutans, S. sanguis, S. salivarius, S. mitior, S. constellatus, S. intermedius, S. anginosus, S. milleri, S. iniae, S. pneumoniae, and combinations thereof.

Preferred, non-limiting examples of such Staphylococcus sp. are those selected from the group consisting of S. aureus, S. epidermidis, and combinations thereof.

Preferred, non-limiting examples of such fungi are those selected from the group consisting of P. ovale, P. versicolor, M. furfur, T. beigelii, B. capitatus, P. marneffei, C. neoformans, S. prolificans, S. shenkii, Epidermophyton floccosum, Microsporum canis, Candida sp., Trichophyton sp., and combinations thereof.

Preferred, non-limiting examples of such Candida sp. are those selected from the group consisting of C. albicans, C. cruzii, C. krusei, C, glabrata, C. guillermondii, C. inconspicua, C. parapsilosis, C. tropicalis, and combinations thereof.

Preferred, non-limiting examples of such Trichophyton sp. are those selected from the group consisting of T. rubrum, T. mentagrophytes, T. tonsurans, T. violaceum, and combinations thereof.

Preferred, non-limiting examples of such molds are those selected from the group consisting of Aspergillus sp., B. dermatitidis, P. brasiliensis, and combinations thereof.

Preferred, non-limiting examples of such Aspergillus sp. are those selected from the group consisting of A. flavus, A. fumigates, A. niger, and combinations thereof.

Several specific skin or hair disorders may also be treated according to the present inventive methods. Exemplary among these skin disorders are seborrheic dermatitis, itchy, flaky scalp conditions, including stubborn dandruff, Pityrosporum infections, pityriasis versicolor, tinea pedis, tinea cruris, tinea corporis, cutaneous candidiasis, and combinations thereof. Other skin disorders known to those of ordinary skill in the art as effectively treatable by a shampoo are further contemplated as within the scope of the present inventive subject matter.

In a preferred embodiment, the skin disorder to be treated according to the present inventive methods is seborrheic dermatitis. Seborrheic dermatitis is a disorder of the scalp differing from simple dandruff by the presence of erythema as a sign of inflammation, by the greater degree of scaling with occasional itching and burning, and by the occurrence of eczematous changes to other body sites. It can occur in the form of patches, but more frequently affects the whole scalp. Further, it can afflict an area beyond the hairline, including the forehead, around the neck, and the ears. In severe cases, the scalp can have a secondary infection, resulting in a spongy consistency, vesicle, crust formation, and weeping.

Seborrheic dermatitis frequently occurs even in infancy and usually remits spontaneously at an age of 8-12 months. The scalp changes such as erythema, scaling, and occasionally vesicles and crusts in infants can regress spontaneously within a few weeks, intermittently reoccur, or persist during the entire childhood. They are frequently combined with a similar process around the eyelids, nose, and ears. Later, the condition usually occurs after puberty and can last for the whole life or even increase in strength.

Further, the present inventive methods provide antimycotic activity against Pityrosporum strains. Pityrosporum yeasts and fungi are believed to be the cause of seborrheic dermatitis.

In another preferred embodiment, the skin disorder to be treated according to the present inventive methods is normal dandruff. Normal dandruff is characterized by a clinically non-inflammatory scaling of the scalp occurring in a large percentage of the population. Dandruff causes an itchy, irritated scalp and flaking skin. Dandruff is caused by an acceleration of the scalp-cell renewal process when the body is fighting off Pityrosporum ovale, causing dead cells to fall of faster.

In another preferred embodiment, the skin disorder to be treated according to the present inventive methods is Pityriasis versicolor, a known superficial, non-inflammatory skin fungus disorder on the trunk.

### Methods of Production

The present inventive subject matter further relates to a process for preparing a storage-stable topical pharmaceutical shampoo, said process comprising:
1) preparing an aqueous phase comprising about 40 to about 90% by weight of the overall weight of the composition of water and a first surfactant at a temperature of about 73 to about 93° C.,
2) cooling said aqueous phase to a temperature of about 44 to about 65° C. while mixing;
3) adding a first surface conditioning agent and at least one chelating agent to said aqueous phase one at a time while mixing until said aqueous phase has a uniform appearance;
4) cooling said aqueous phase to a temperature of about 22 to about 42° C.;
5) preparing an active ingredient solution comprising a second surfactant and about 0.5-8% of the overall weight of the composition of an active ingredient comprising an antimicrobial agent or a pharmaceutically acceptable salt thereof at a temperature of about 22 to about 42° C.;
6) adding said active ingredient solution to said aqueous phase;
7) adding sufficient amounts of at least one pH modifier to provide said aqueous phase with a pH of about 5.5 to about 7.0; and
8) recovering a storage-stable topical pharmaceutical shampoo.

In a preferred embodiment of the present inventive subject matter, the aqueous phase is prepared according to said process step 1) by further adding at least one thickening agent and a second surface conditioning agent to said aqueous phase prior to addition of said first surfactant, and mixing until all ingredients are melted. In a preferred embodiment, the first surfactant in said aqueous phase is an anionic surfactant.

In another preferred embodiment of the present inventive subject matter, samples of the aqueous phase are collected and slowly poured back into the aqueous phase after the aqueous phase is cooled to a temperature of about 44 to about 65° C. but before the first surface conditioning agent and at least one chelating agent are added to the aqueous phase. As these samples are poured back into the aqueous phase, the solution is observed for unhydrated particles. If unhydrated particles are observed, then said aqueous phase is mixed for at least a further fifteen minutes. The process of collecting samples of the aqueous phase, pouring the sample back into the aqueous phase, observing the solution for unhydrated particles, and further mixing the aqueous phase is repeared until no unhydrated particles are observed.

In a further preferred embodiment, the active ingredient solution is prepared by mixing for at least 70 minutes until dissolution of the active ingredient is complete. No foam should be generated during the preparation of the active ingredient solution. In a further preferred embodiment, the second surfactant in said aqueous phase is an amphoteric surfactant.

Once each of the aqueous phase and the active ingredient solution have been separately prepared, the active ingredient solution is added to the aqueous phase and maintained at a temperature of about about 22 to about 42° C. and mixed for at least fifteen minutes.

Further contemplated as within the scope of the present inventive subject matter are pharmaceutical compositions produced according to the above-described process. If produced according to the present inventive process, these compositions exhibit chemical and physical stability suitable for topical administration.

In further preferred embodiments, the at least one pH modifier is selected from the group consisting of sodium hydroxide, citric acid, and a mixture thereof. Prior to addition of the pH modifier, the pH of the aqueous phase will be tested to determine which pH modifier should properly be added to obtain the desired pH.

The compositions produced according to these processes can be placed in a suitable containment vessel comprising a product contact surface composed of a material selected from the group consisting of glass, plastic, steel, stainless steel, aluminum, Teflon, polymeric structure, ceramic structure, alloys, and mixtures thereof. These containment vessels are used to facilitate manufacturing, handling, processing, packaging, storage, and administration of said composition.

### Dosage

Appropriate dosage levels for the active antimicrobial agents contemplated in the present inventive subject matter are well known to those of ordinary skill in the art. Dosage levels on the order of about 0.001 mg to about 5,000 mg per kilogram body weight of the active therapeutic compounds or compositions are known to be useful in the treatment of the diseases, disorders, and conditions contemplated in the present inventive subject matter. Typically, this effective amount of the active antimicrobial agents will generally comprise from about 0.1 mg to about 100 mg per kilogram of patient body weight per day.

If desired, other therapeutic agents can be employed in conjunction with those provided by the present inventive subject matter. The amount of active ingredients that may be combined with the carrier materials to produce a single dosage form will vary depending upon the host treated, the nature of the disease, disorder, or condition, and the nature of the active ingredients.

The present inventive compositions may be topically applied to the head or scalp from one to seven times per week. Preferably, the present inventive compositions are applied to the head or scalp two or three times a week, with at least one day between applications. A set application schedule is recommended in this regard.

It is understood, however, that a specific dose level for any particular patient will depend upon a variety of factors well known in the art, including the activity of the specific compound employed; the age, body weight, general health, sex and diet of the patient; the time of administration; the rate of excretion; drug combination; the severity of the particular disorder being treated; and the form of administration. One of ordinary skill in the art would appreciate the variability of such factors and would be able to establish specific dose levels using no more than routine experimentation.

The optimal pharmaceutical formulations will be determined by one skilled in the art depending upon considerations such as the particular drug or drug combination and the desired dosage. See, for example, “Remington's Pharmaceutical Sciences”, 18th ed. (1990, Mack Publishing Co., Easton, Pa. 18042), pp. 1435-1712, the disclosure of which is hereby incorporated by reference. Such formulations may influence the physical state, stability, rate of in vivo release, and rate of in vivo clearance of the therapeutic agents.

In one preferred application regimen, a sufficient amount of the present inventive antimicrobial shampoo to produce an abundant lather is applied to wetted hair and scalp. The shampoo is allowed to remain on the scalp for 1 to 5 minutes, after which it is rinsed from the hair and scalp.

In another preferred application regimen, a sufficient amount of the present inventive antimicrobial shampoo to produce an abundant lather is applied to wetted hair and scalp. This first application is massaged over the entire scalp for approximately 2-5 minutes and then rinsed. A second application of the antimicrobial shampoo is applied immediately after the first rinsing and again massaged over the entire scalp for approximately 2-5 minutes, then rinsed.

The present inventive shampoos are prepared in a manner known per se by mixing together the individual components and further processing, if necessary.

### EXAMPLES

The following examples are illustrative of the present inventive subject matter and are not intended to be limitations thereon. All polymer molecular weights are mean average molecular weights. All percentages are based on the percent by weight of the final delivery system or formulation prepared unless otherwise indicated and all totals equal 100% by weight.

### Example 1

The following example illustrates a label claim formula for an antimicrobial shampoo of the present inventive subject matter:

| | % W/W |
|---|---|
| Ciclopirox Olamine | 2.00 |
| Benzyl Alcohol | 2.50 |
| Butylene Glycol | 2.00 |
| Cetrimonium Chloride | 0.60 |
| Citric Acid | 0.70 |
| Cocamidopropyl Betaine | 2.10 |
| Edetate Disodium | 0.10 |
| Hydroxypropyl Methylcellulose 2910 | 0.70 |
| Lauramide MEA | 2.75 |
| PEG-75 Lanolin | 1.00 |
| Purified Water | 70.35 |
| Sodium hydroxide | q.s. pH |
| | about 6.5 |
| TEA-Lauryl Sulfate | 15.2 |
| | 100.0% |

This final antimicrobial shampoo formulation can be prepared as follows:

1. An aqueous phase is prepared by mixing the Hydroxypropyl Methylcellulose 2910 in purified water for about 10 minutes at a temperature of 83±2° C. The PEG-75 Lanolin and Lauramide MEA are then slowly added one at a time to this mixture and then mixed until all ingredients are melted. Temperature is maintained at 83±2° C. The TEA-Lauryl Sulfate is then slowly added to this mixture over a minimum of 15 minutes. After mixing, the temperature of the aqueous phase is lowered to 54±2° C. and then mixed for 15 minutes. Samples of the aqueous phase are then repeatedly taken and returned to the aqueous phase, followed by mixing for at least 15 minutes, until no unhydrated particles are observed. The Butylene Glycol, Cetrimonium Chloride, Edetate Disodium, and Citric Acid are then slowly added one at a time to this mixture and then mixed for at least 10 minutes until a uniform appearance is produced while temperature is maintained at 54±2° C. After mixing, the temperature of the aqueous phase is lowered to 32±2° C.

2. A sodium hydroxide solution is prepared by slowly adding the Sodium Hydroxide to purified water while stirring. The sodium hydroxide solution is then added to the aqueous phase, and mixed for about 15 minutes. Temperature is maintained at 32±2° C.

3. An active ingredient solution is prepared by adding the Benzyl Alcohol to the Cocamidopropyl Betaine at a temperature of 83±2° C. and then mixing for about 30 minutes, while avoiding the generation of foam. The Ciclopirox Olamine is then added to the mixture and mixed for a minimum of 40 minutes until dissolution is complete. The active ingredient solution is then added to the aqueous phase at a temperature of 32±2° C. The aqueous solution is then mixed for about 15 minutes until uniform in appearance. The pH of the aqueous solution is then tested. If the pH is below 6.2, a sodium hydroxide solution is added and mixed until the pH is between 6.2 and 6.8. If the pH is above 6.8, a citric acid solution is added and mixed until the pH is between 6.2 and 6.8.

### Example 2

The following example illustrates the manufacturing formula, rather than the label claim formula, of the antimicrobial shampoo of Example 1:

| | % W/W |
|---|---|
| Ciclopirox Olamine | 2.00 |
| Benzyl Alcohol | 2.50 |
| Butylene Glycol | 2.00 |
| Cetrimonium Chloride | 2.00 |
| (30% w/w ingredient) | |
| Citric Acid | 0.70 |
| Cocamidopropyl Betaine | 6.00 |
| (35% w/w ingredient) | |
| Edetate Disodium | 0.10 |
| Hydroxypropyl Methylcellulose 2910 | 0.70 |
| Lauramide MEA | 2.75 |
| PEG-75 Lanolin | 1.00 |
| Purified Water | 42.22 |
| Sodium hydroxide | 0.03 |
| TEA-Lauryl Sulfate | 38.00 |
| (40% w/w ingredient) | |
| | 100.0% |

The amounts of cetrimonium chloride, cocamidopropyl betaine, and TEA-lauryl sulfate used in this formulation represent amounts of a commercially available pre-mix for each of these components. The pre-mix of each of these ingredients contains water therein-they are not added to the process as pure components. Rather, these pre-mixes each contain the indicated amount of the respective ingredients. These amounts of pre-mixes are used for manufacturing purposes only and are not indicative of the amounts of these components (also including water) in the final formulation.

Further, this shampoo is prepared according to the process described above for Example 1.

### Example 3

A patient is suffering from seborrheic dermatitis. An antimicrobial shampoo composition of the present inventive subject matter is topically administered to the patient. It would be expected that the patient would improve his/her condition or recover.

### Example 4

A patient is suffering from dandruff. An antimicrobial shampoo composition of the present inventive subject matter is topically administered to the patient. It would be expected that the patient would improve his/her condition or recover.

The inventive subject matter being thus described, it will be apparent that the same may be modified or varied in many ways. Such modifications and variations are not to be regarded as a departure from the spirit and scope of the inventive subject matter, and all such modifications and variations are intended to be included within the scope of the following claims.

1. A storage-stable topical pharmaceutical shampoo comprising:
about 0.5-8% by weight of an active ingredient comprising an antimicrobial agent or a pharmaceutically acceptable salt thereof;
about 0.5-30% by weight of at least one surfactant selected from the group consisting of an amphoteric surfactant, an anionic surfactant, and mixtures thereof;
about 0.01-1% by weight of at least one chelating agent;
about 40-90% by weight of purified water; and
sufficient amounts of at least one pH modifier selected from the group consisting of pharmaceutically acceptable acids, bases, and mixtures thereof to provide the pharmaceutical shampoo with an overall pH of about 3.0 to about 8.0;
wherein said active ingredient maintains a concentration of degradation product(s) less than about 5% of the starting concentration of said active ingredient.

2. The shampoo of claim 1, wherein said antimicrobial agent is a compound having the formula I: 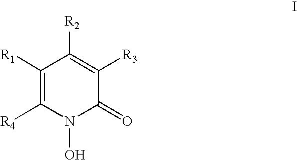
or a pharmaceutically acceptable salt thereof, wherein:
R₁, R₂, and R₃, which are identical or different, are H or alkyl having 1 to 4 carbon atoms, and R₄ is a saturated hydrocarbon radical having 6 to 9 carbon atoms or a radical of formula II: 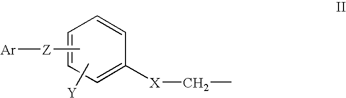
where:
X is S or O;
Y is selected from the group consisting of H, 1 or 2 identical halogen atoms, and a mixture of 2 different halogen atoms;
Z is selected from the group consisting of a single bond and a bivalent radical comprising O, S, CR₂ where R₂ is H or (C₁-C₄)-alkyl, or from 2 to 10 carbon atoms linked in the form of a chain, which optionally further comprises one or more of the following:
(i) a carbon-carbon double bond, or
(ii) O, S, or a mixture thereof, wherein if 2 or more O or S atoms or a mixture thereof are present, each O or S atom is separated by at least 2 carbon atoms; and,
in any of the foregoing bivalent radicals, free valences of the carbon atoms of said bivalent radical are saturated by H, (C₁-C₄)-alkyl, or a mixture thereof; and
Ar is an aromatic ring system having one or two rings that can be substituted by one, two, or three radicals, which may be identical or different, which are selected from the group consisting of halogen, methoxy, (C₁-C₄)-alkyl, trifluoromethyl, or trifluoromethoxy.

3. The shampoo of claim 2, wherein said antimicrobial agent is selected from the group consisting of 6-[4-(4-chlorophenoxy)-phenoxymethyl]-1-hydroxy-4-methyl-2-pyridone, 1-hydroxy-4-methyl-6-cyclohexyl-2-pyridone, 1-hydroxy-4-methyl-6-(2,4,4-trimethylpentyl)-2-pyridone, a pharmaceutically acceptable salt thereof, and a mixture thereof.

4. The shampoo of claim 3, wherein said antimicrobial agent is 1-hydroxy-4-methyl-6-cyclohexyl-2-pyridone or a pharmaceutically acceptable salt thereof.

5. The shampoo of claim 1, wherein said antimicrobial agent possesses anti-inflammatory properties.

6. The shampoo of claim 1, wherein said antimicrobial agent possesses activity against microbes selected from the group consisting of gram positive bacteria, gram negative bacteria, funguses, molds, viruses, and combinations thereof.

7. The shampoo of claim 1, wherein said active ingredient has a concentration of degradation product(s) less than about 2% of the starting concentration of said active ingredient.

8. The shampoo of claim 1, comprising about 1 to about 5% by weight of said active ingredient.

9. The shampoo of claim 1, wherein said shampoo possesses anti-inflammatory properties.

10. The shampoo of claim 1, wherein said at least one surfactant comprises at least one amphoteric surfactant and at least one anionic surfactant.

11. The shampoo of claim 10, wherein said amphoteric surfactant is cocoamidopropyl betaine.

12. The shampoo of claim 10, wherein said anionic surfactant is triethylamine lauryl sulfate.

13. The shampoo of claim 1, comprising about 12-22% by weight of said at least one surfactant.

14. The shampoo of claim 1, wherein said chelating agent is disodium edetate.

15. The shampoo of claim 1, wherein said shampoo has a pH of about 5.5 to about 7.0.

16. The shampoo of claim 15, wherein said shampoo has a pH of about 6.5.

17. The shampoo of claim 1, wherein said pH modifier is selected from the group consisting of sodium hydroxide, citric acid, and a mixture thereof.

18. The shampoo of claim 1, further comprising about 0.1-5% by weight of at least one conditioning agent.

19. The shampoo of claim 18, wherein said conditioning agent affects the physical properties of a surface to which said shampoo is applied.

20. The shampoo of claim 19, wherein said surface is selected from the group consisting of populated hair, hair follicles, a surface contiguous to or in close proximity to hair, sweat glands, sebaceous glands, and combinations thereof.

21. The shampoo of claim 18, wherein said conditioning agent is a hair conditioning agent.

22. The shampoo of claim 21, wherein said hair conditioning agent is selected from the group consisting of a silicone compound, a quaternary ammonium compound, a fatty compound, a lanolin or a derivative thereof, and mixtures thereof.

23. The shampoo of claim 22, wherein said hair conditioning agent is a mixture of cetrimonium chloride and ethoxylated polyethylene glycol lanolin.

24. The shampoo of claim 21, comprising about 0.5-2.5% by weight of said at least one hair conditioning agent.

25. The shampoo of claim 1 which further comprises an additional ingredient selected from the group consisting of a humectant, inorganic salt, fragrance, dye, hair colorant, foam stabilizer, preservative, water softening agent, and mixtures thereof.

26. The shampoo of claim 1 which further comprises a thickener selected from the group consisting of methylcellulose, hydroxybutyl methylcellulose, hydroxypropylcellulose, hydroxypropyl methylcellulose, hydroxyethyl ethylcellulose, hydroxyethylcellulose, di(hydrogenated tallow)phthalic acid amide, crosslinked maleic anhydride-methyl vinyl ether copolymer, guar gum, xanthan gum, gum arabic, and mixtures thereof.

27. A method of treating a skin or hair disorder in a mammal, comprising administering to skin or hair of a mammal in need thereof a therapeutically effective amount of a storage-stable topical pharmaceutical shampoo comprising:
about 0.5-8% by weight of an active ingredient comprising an antimicrobial agent or a pharmaceutically acceptable salt thereof;
about 0.5-30% by weight of at least one surfactant selected from the group consisting of an amphoteric surfactant, an anionic surfactant, and mixtures thereof;
about 0.01-1% by weight of at least one chelating agent;
about 40-90% by weight of purified water; and
sufficient amounts of at least one pH modifier selected from the group consisting of pharmaceutically acceptable acids, bases, and mixtures thereof to provide the pharmaceutical shampoo with an overall pH of about 3.0 to about 8.0;
wherein said active ingredient maintains a concentration of degradation product(s) less than about 5% of the starting concentration of said active ingredient.

28. The method of claim 27, wherein said skin or hair is selected from the group consisting of populated hair, hair follicles, a surface contiguous to or in close proximity to hair, sweat glands, sebaceous glands, and combinations thereof.

29. The method of claim 27, wherein the administration of said shampoo cleans hair or scalp of said mammal.

30. The method of claim 27, wherein said skin or hair is natural or synthetic skin or hair.

31. The method of claim 27, wherein the administration of said shampoo reduces the number of microbes on hair or scalp of said mammal.

32. The method of claim 31, wherein said microbes are pathogenic.

33. The method of claim 31, wherein said microbes are selected from the group consisting of bacteria, funguses, molds, viruses, and combinations thereof.

34. The method of claim 33, wherein said bacteria is selected from the group consisting of gram positive bacteria, gram negative bacteria, and combinations thereof.

35. The method of claim 34, wherein said gram positive bacteria are selected from the group consisting of Streptococcus sp., Micrococcus sp., Staphylococcus sp., Bacillus sp., and combinations thereof.

36. The method of claim 35, wherein said Streptococcus sp. are selected from the group consisting of S. viridans, S. agalactiae, S. pyogenes, S. faecalis, S. durans, S. faecium, S. mutans, S. sanguis, S. salivarius, S. mitior, S. constellatus, S. intermedius, S. anginosus, S. milleri, S. iniae, S. pneumoniae, and combinations thereof.

37. The method of claim 35, wherein said Staphylococcus sp. are selected from the group consisting of S. aureus, S. epidermidis, and combinations thereof.

38. The method of claim 33, wherein said fungus is selected from the group consisting of P. ovale, P. versicolor, M. furfur, T. beigelii, B. capitatus, P. marneffei, C. neoformans, S. prolificans, S. shenkii, Epidermophyton floccosum, Microsporum canis, Candida sp., Trichophyton sp., and combinations thereof.

39. The method of claim 38, wherein said Candida sp. are selected from the group consisting of C. albicans, C. cruzii, C. krusei, C, glabrata, C. guillermondii, C. inconspicua, C. parapsilosis, C. tropicalis, and combinations thereof.

40. The method of claim 38, wherein said Trichophyton sp. are selected from the group consisting of T. rubrum, T. mentagrophytes, T. tonsurans, T. violaceum, and combinations thereof.

41. The method of claim 33, wherein said mold is selected from the group consisting of Aspergillus sp., B. dermatitidis, P. brasiliensis, and combinations thereof.

42. The method of claim 41, wherein said Aspergillus sp. are selected from the group consisting of A. flavus, A. fumigates, A. niger, and combinations thereof.

43. The method of claim 27, wherein said skin or hair disorder is selected from the group consisting of dandruff, seborrheic dermatitis, itchy flaky scalp conditions, pityriasis versicolor, tinea pedis, tinea cruris, tinea corporis, cutaneous candidiasis, and combinations thereof.

44. A storage-stable topical pharmaceutical shampoo comprising:
about 0.5-8% by weight of an active ingredient comprising ciclopirox or a pharmaceutically acceptable salt thereof;
about 0.5-30% by weight of at least one surfactant selected from the group consisting of an amphoteric surfactant, an anionic surfactant, and mixtures thereof;
about 0.01-1% by weight of at least one chelating agent;
about 40-90% by weight of purified water; and
sufficient amounts of at least one pH modifier selected from the group consisting of citric acid, sodium hydroxide, and mixtures thereof to provide the pharmaceutical shampoo with an overall pH of about 5.5 to about 7.0;
wherein said active ingredient maintains a concentration of degradation product(s) less than about 5% of the starting concentration of said active ingredient.

45. The shampoo of claim 44, further comprising about 0.1-5% by weight of at least one hair conditioning agent selected from the group consisting of cetrimonium chloride, ethoxylated polyethylene glycol lanolin, and mixtures thereof.

46. The shampoo of claim 44, wherein said active ingredient has a concentration of degradation product(s) less than about 2% of the starting concentration of said active ingredient.

47. The shampoo of claim 44, wherein said at least one surfactant is selected from the group consisting of cocamidopropyl betaine, triethylamine lauryl sulfate, and mixtures thereof.

48. A method of treating a skin disorder in a mammal, comprising administering to a mammal in need thereof a therapeutically effective amount of a storage-stable topical pharmaceutical shampoo comprising:
about 0.5-8% by weight of an active ingredient comprising ciclopirox or a pharmaceutically acceptable salt thereof;
about 0.5-30% by weight of at least one surfactant selected from the group consisting of an amphoteric surfactant, an anionic surfactant, and mixtures thereof;
about 0.01-1% by weight of at least one chelating agent;
about 40-90% by weight of purified water; and
sufficient amounts of at least one pH modifier selected from the group consisting of citric acid, sodium hydroxide, and mixtures thereof to provide the pharmaceutical shampoo with an overall pH of about 5.5 to about 7.0;
wherein said active ingredient maintains a concentration of degradation product(s) less than about 5% of the starting concentration of said active ingredient.

49. A process for preparing a storage-stable topical pharmaceutical shampoo, said process comprising:
1) preparing an aqueous phase comprising about 40 to about 90% by weight of the overall weight of the composition of water and a first surfactant at a temperature of about 73 to about 93° C.,
2) cooling said aqueous phase to a temperature of about 44 to about 65° C. while mixing;
3) adding a first surface conditioning agent and at least one chelating agent to said aqueous phase one at a time while mixing until said aqueous phase has a uniform appearance;
4) cooling said aqueous phase to a temperature of about 22 to about 42° C.;
5) preparing an active ingredient solution comprising a second surfactant and about 0.5-8% of the overall weight of the composition of an active ingredient comprising an antimicrobial agent or a pharmaceutically acceptable salt thereof at a temperature of about 22 to about 42° C.;
6) adding said active ingredient solution to said aqueous phase;
7) adding sufficient amounts of at least one pH modifier to provide said aqueous phase with a pH of about 5.5 to about 7.0; and
8) recovering a storage-stable topical pharmaceutical shampoo.

50. The process of claim 49, wherein said first surfactant is an anionic surfactant.

51. The process of claim 49, wherein said process step 1) further comprises adding at least one thickening agent and a second surface conditioning agent to said aqueous phase prior to addition of said first surfactant, and mixing until all ingredients are melted.

52. The process of claim 49, wherein between said step 2) and said step 3), samples of said aqueous phase are collected and slowly poured back into said aqueous phase while observing for unhydrated particles.

53. The process of claim 52, wherein said aqueous phase is mixed for at least a further fifteen minutes if unhydrated particles are observed until said unhydrated particles are no longer observed.

54. The process of claim 49, wherein said second surfactant is an amphoteric surfactant.

55. The process of claim 49, wherein said active ingredient solution is prepared by mixing for at least 70 minutes until dissolution of said active ingredient is complete without generating foam during said preparation.

56. The process of claim 49, wherein said aqueous phase is mixed for at least fifteen minutes after said active ingredient solution is added thereto.

57. The process of claim 49, wherein said at least one pH modifier is selected from the group consisting of sodium hydroxide, citric acid, and a mixture thereof.
